(19) United States
(12) Patent Application Publication
(10) Pub. No.: US 2016/0256611 A1
(43) Pub. Date: Sep. 8, 2016

(54) Drug Delivery Device

(71) Applicant: MicroVention, Inc., Tustin, CA (US)

(72) Inventor: Matthew J. Fitz, Vista, CA (US)

(73) Assignee: MicroVention, Inc., Tustin, CA (US)

(21) Appl. No.: 15/061,840

(22) Filed: Mar. 4, 2016

Related U.S. Application Data
(60) Provisional application No. 62/128,386, filed on Mar. 4, 2015.

(51) Int. Cl. A61L 31/16; A61L 31/04; A61L 31/06; A61L 31/02; A61L 31/14

(52) U.S. Cl. CPC A61L 31/16; A61L 31/026; A61L 31/145; A61L 31/06; A61L 31/048; A61L 31/022; A61L 2300/416

(57) ABSTRACT

A drug delivery device, method of making a drug delivery device, and method of using a drug delivery device are described. The drug delivery device may be used to treat a target area within a patient's vasculature and comprises a shell, agent, port, and an optional seal. The agent may be any number of compounds, including but not limited to a therapeutic, anti-cancer compound.

### RELATED APPLICATIONS

This application claims priority to U.S. Provisional Application Ser. No. 62/128,386 filed Mar. 4, 2015 entitled Drug Delivery Device, which is hereby incorporated herein by reference in its entirety.

### BACKGROUND OF THE INVENTION

Diseases such as cancer or other tumors may be treated by advancing a catheter within in a blood vessel to a location near the cancer and infusing a chemotherapy drug into the tissue. Another cancer treatment involves fusing small polymer beads into the cancerous tissue such that they become lodged in the tissue and occlude the blood flow to it. Another cancer treatment known as transarterial chemoembolization (TACE) involves infusing polymer beads with chemotherapy drugs, such as irinotecan or doxorubicin, and injecting them near the cancerous tissue. Yet another treatment option infuses radioactive beads or pellets made from materials such as yttrium-90, palladium-103, or cobalt-60 near the tumor.

The following embodiments disclose different devices and methods to treat diseases.

### SUMMARY OF THE INVENTION

In one embodiment a drug delivery device comprising a shell and an agent disposed within the shell is described.

In another embodiment a drug delivery device comprising a shell, agent disposed within in a shell, and a port is described.

In another embodiment a drug delivery device comprising a shell, an agent disposed within the shell, a port, and a seal is described.

In another embodiment a drug delivery device comprising a shell, an agent disposed within the shell, a port, and a degradable seal is described.

In another embodiment a drug delivery device comprising a radiopaque shell is described.

In another embodiment a drug delivery device includes an agent mixed with another compound to control the diffusion rate of the agent.

In another embodiment a drug delivery device includes an agent, wherein the concentration of the agent is adjusted to control the diffusion rate of the agent.

In another embodiment, one or more drug delivery devices may be arranged on a frame and filled by an automated, computer controlled process.

In another embodiment a drug delivery device includes a shell and an anti-cancer, therapeutic agent disposed within the shell.

In another embodiment, one or more drug delivery devices comprising a shell and an agent disposed within a shell are transmitted to a treatment site and delivered to a target area.

In another embodiment, a therapeutic procedure is carried out by using a drug delivery device with a therapeutic agent therein, and delivering said drug delivery device to a target area of the vasculature, where said agent is released at the target area.

In another embodiment, a cancer treatment is carried out by using a drug delivery device with a therapeutic, anti-cancer agent therein, and delivering said drug delivery device to a target area of the vasculature, wherein said agent is released at the target area.

In another embodiment a drug delivery device is comprised of several devices with agents therein connected together.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIGS. 1A and 1B illustrate a drug delivery device according to one embodiment.

FIGS. 2, 3A, and 3B illustrate a drug delivery device according to another embodiment.

FIG. 4 illustrates delivery of a drug delivery device to cancerous tissue.

FIG. 5 illustrates delivery of several drug delivery devices to a stent deployed near cancerous tissue.

FIG. 6 illustrates drug delivery devices in a syringe for delivery into a delivery catheter or directly into cancerous tissue.

FIG. 7A illustrates a frame in which a drug delivery device is composed.

FIG. 7B illustrates a filling machine that fills a drug delivery device with a treatment agent.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention is generally directed to a relatively small capsule or shell containing a cancer-treating agent. A plurality of these capsules or shells are delivered to cancerous tissue for causing treatment. The embodiments disclosed herein may comprise a shell portion, a cancer treatment agent located within the shell portion, and, in some embodiments, a seal that seals the shell portion and helps to at least partially contain the agent.

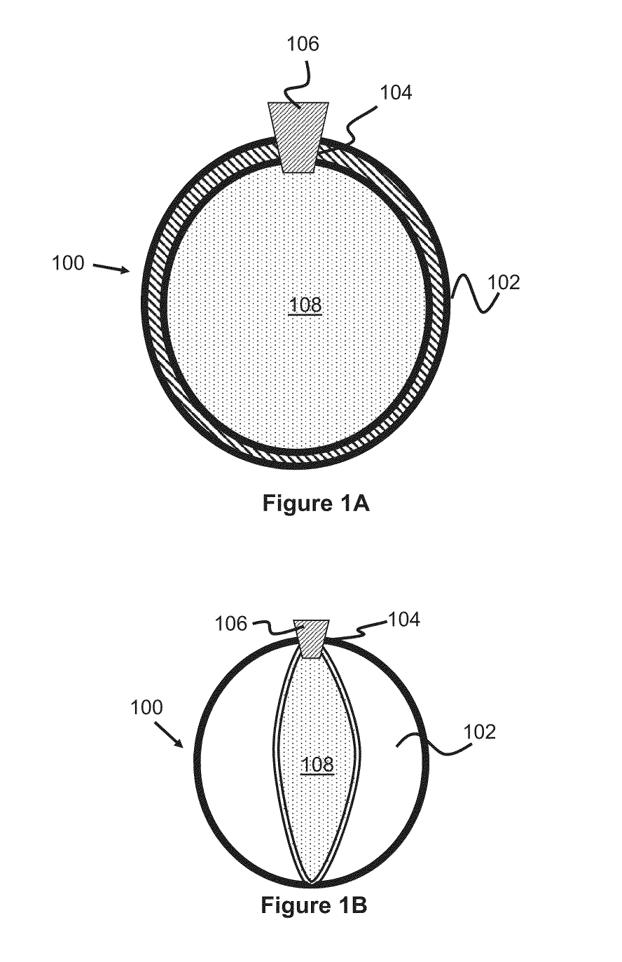

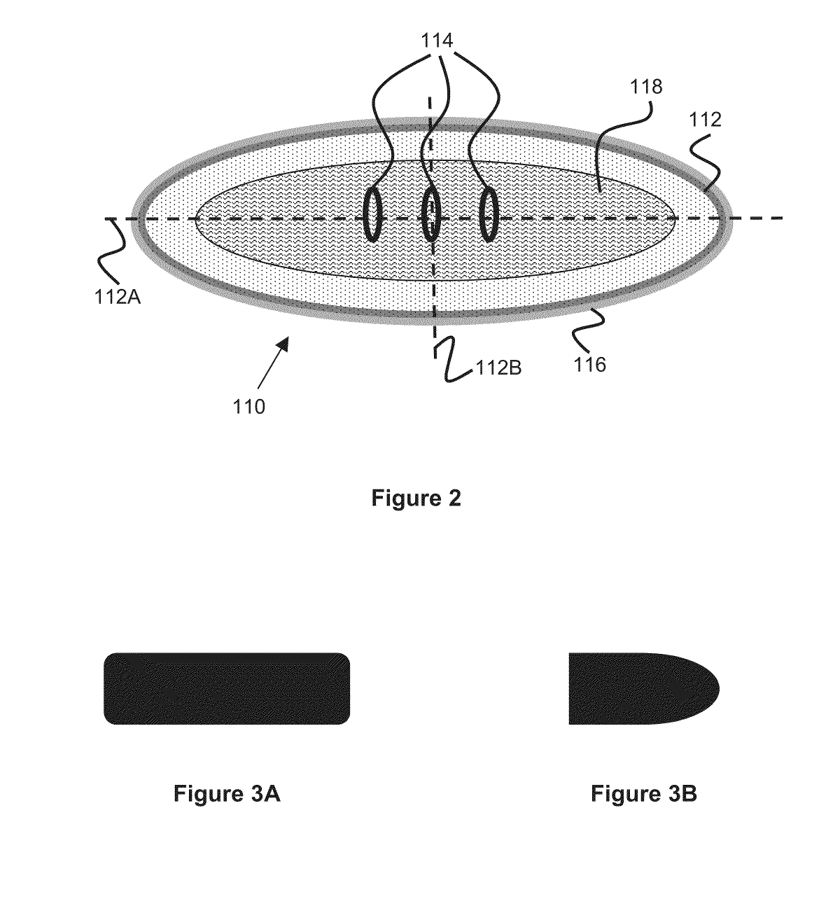

FIGS. 1A, 1B, and 2 illustrate an embodiment of a drug delivery device according to the present invention. Referring first to FIG. 1, a device 100 is illustrated, comprising a spherical, hollow shell 102, an aperture or port 104 into an interior of the shell, a seal 106 that seals the port 104 closed, and an agent 108 that treats cancerous tissue. One or more of these devices can be used for treatment by delivering the devices 100 into or near the cancerous tissue. The seal 106 may degrade, rupture, weaken, or otherwise cause the port 104 to allow passage of materials through it, allowing the treatment agent 108 to escape the shell 102 and dissipate into the cancerous tissue. Hence, known cancer treatment agents can be used without the need to alter their chemical structure, as is typically needed with previously known polymer beads, so as to allow the agents to bind to and later release from the polymer of the beads.

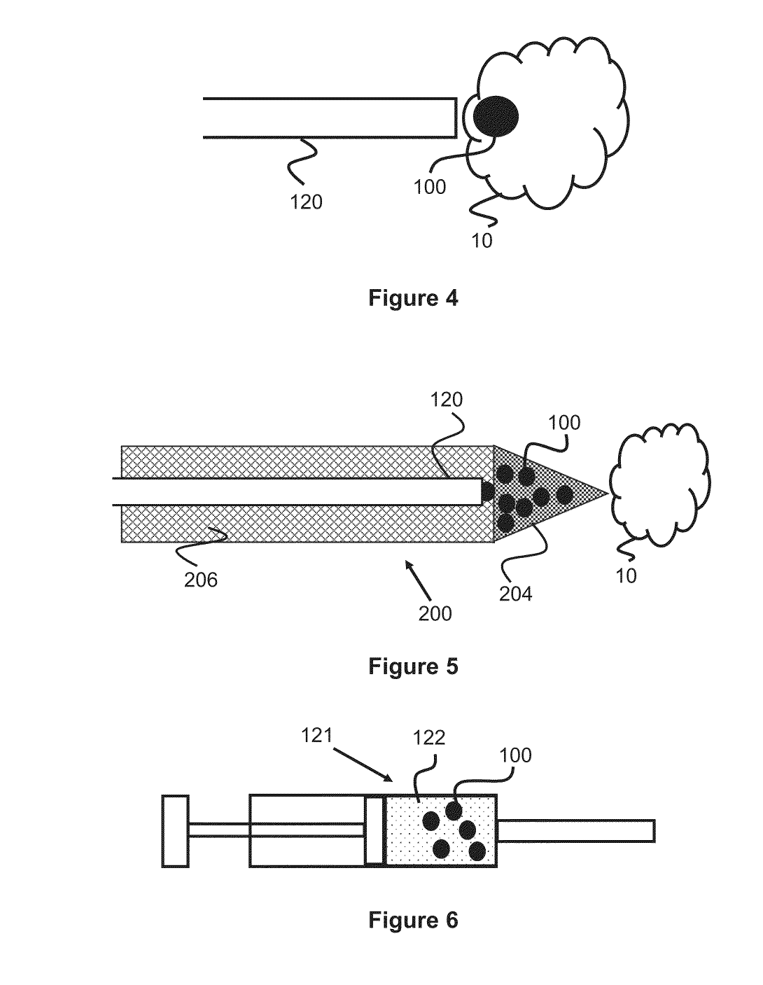

One delivery example is illustrated in FIG. 4, a tubular delivery device 120 is used for delivery into or near the cancerous tissue 10. In this respect, the drug delivery devices become embedded or contained in the tissue 10, allowing the treatment agent 108 to spread into the tissue 10. The tubular delivery device 120 can be a needle syringe, a catheter, a syringe needle injecting into a catheter, or a similar delivery device.

Another delivery example is illustrated in FIG. 5, in which a filter stent 200, having a cylindrical stent portion and a distally-attached filter portion 204 is filled with one or more of the drug delivery devices 100. The filter stent 200 is preferably delivered within a blood vessel upstream and preferably feeding the cancerous tissue 10. The delivery catheter 120 is advanced into the filter stent 200 and a guidewire or pusher member within the catheter is distally advanced so as to push out the one or more devices 100 into the filter portion 204 of the filter stent 200. In this respect, the devices 100 can occlude the blood vessel supplying the cancerous tissue 10 while also delivering treatment agents 108 to the tissue 10. Additionally, embolic coils may also be delivered to the filter portion 204 to further enhance occlusion of the vessel. Additional details and filter stent embodiments can be found in U.S. application Ser. No. 15/053,970 filed Feb. 25, 2016 and entitled Stent and Filter, the contents of which is herein incorporated by reference.

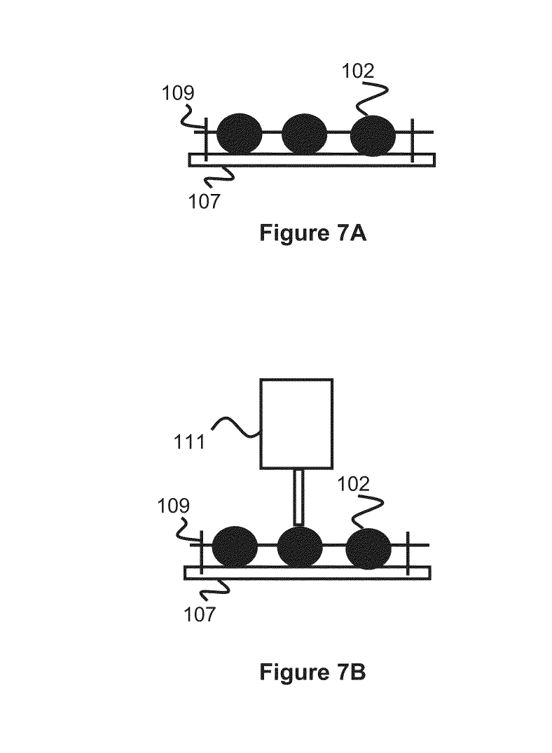

The shell 102 may be composed of a biocompatible metal, such as a palladium alloy, and can be formed by laser cutting a solid portion of material (e.g., cutting away two half-portions of the shell and then adhering or welding them together to form a single shell), casting, or injection molding. The outer surface of the shell 102 and the inner cavity can take a variety of different shapes, such as spherical or ovaloid. The port 104 can be formed as part of the molding or casting process of the shell 102, as part of the laser cutting process, or can be formed by drilling after the shell 102 has been formed. In one example, the port 104 has a diameter of about 20 microns and incorporates a taper to allow it to mate to a standard needle (i.e., the port 104 narrows towards the interior of the shell 102). As seen in FIGS. 7A and 7B, the shell 102 of the device 100 can be formed on a single wafer 107 using microfabrication and a series of shells are connected to each other on a precision frame 109 to facilitate insertion into a precision-controlled filling machine 111. In another example, a filling machine 111 fills each shell 102 with approximately 0.01 microliters of agent 108. In this example, the agent is Bevacizumab (Avastin) which may be used to treat brain, colon, kidney, and/or lung cancer. Though agent 108 is shown as completely filling the interior volume of the device 100, the agent may fill only a portion of the device. In one example, the agent fills only a portion of the shell and a biocompatible fluid, such as saline, fills the rest of the shell so that there is no air or other gas present. A concentrated quantity of the agent may be used so that the saline dose not over-dilute the therapeutic dosage. Alternately, a gas may fill the remaining portion of the interior or the shell.

Once filled with the agent 108, the frame containing multiple shells is passed to a second machine that plugs the port 104 with a biodegradable seal 106. In one embodiment, the seal is made from PGLA (poly(lactic-co-glycolic) acid) dissolved in a solvent such as acetone or ethyl acetate to make it injectable through a small gauge needle. The device 100 is then heated to evaporate or dissipate the solvent and thereby solidify the seal 106. A laser can be used to cut the shell(s) away from the holding frame.

The device 100 can then be packaged by a number of techniques, such as in a vial or pouch without fluids (i.e., dry), or a liquid (i.e., wet) that does not degrade the seal such as alcohol or linseed oil. Wet packaging may be desirable in some situations in which a pre-filled syringe 121 (FIG. 6) is used to deliver the device(s) 100. To prepare the device(s) 100 for delivery, the user mixes the device(s) 100 with a delivery carrier 122 such as saline solution, contrast solution, and/or oil. It may be desirable to provide a mixture of different devices 100, possibly incorporating seals 106 with different degradation properties, in order to control the time-release properties of the agent (i.e., some seals 106 may open immediately and some may open at a predetermined time in the future). For example, several larger 1000 micron devices with relatively fast seal degradation (or possibly no seal) are mixed with 200 micron devices to provide an initial bolus of agent followed by a slower, steadier release over, for example, 3-90 days. It is also possible to mix devices with different agents or mix devices from this example with other devices, such as conventional drug-loadable beads, biodegradable, and/or unloaded beads to occlude flow to a tumor. These combinations can be advantageous for providing a cocktail or drugs as is common with chemotherapy procedures.

Once the desired mixture has been determined and the user has loaded the delivery device (e.g., syringe 121) with the appropriate carrier solution 122, an access device such as a microcatheter 120, guide catheter, or balloon is placed near the treatment site. The solution 122 and devices 100 are then infused into the access device by, for example, a syringe 121, pump, or pressure bag. These delivery procedures are an example and other delivery procedures, such as directly delivering the devices 100 via syringe injection, are also possible.

FIG. 2 illustrates an alternate embodiment of a device 110 comprising a shell 112, multiple ports 114, a sealing coating 116, and a major axis 112A and a minor axis 112B. Ports 114 may be located in proximity to each other along one side of the shell 112, can be located on opposite sides of the shell 112, or can be located at a plurality of positioned on the shell.

In one embodiment, one portion of the shell 112 can be flattened (e.g., at a narrow end of the shell 112 or along a side of the shell 112) as seen in the side profile view of FIG. 3B, both ends of the shell 112 can be flattened as seen in FIG. 3A, or one or more flat portions may extend along a side of the shell 112 (i.e., in a direction parallel with the major axis 112A). The size and shape in this example are configured to be more easily pushable through, for example, a catheter with an inner diameter of 0.017″. Specifically, the flattened end faces proximally in a delivery catheter/device, which allows pusher or guidewire to more easily push the device(s) 110 in a distal direction and out the distal end of the delivery catheter.

The minor axis, in one example, is about 350 microns and the major axis is about 500 microns. The shell 112 is composed of a polymer material such as ABS (acrylonitrile butadiene styrene) or a photopolymer such as MED610 and, in one example, may be formed using 3D printing techniques. The ports 114 are approximately 5-30 microns each and may be formed during the 3D printing process, or by laser or mechanical cutting. After manufacture, as previously described, the assembly is coated with a biodegradable polymer sealing coating 116 such as PGLA or a biodegradable hydrogel such as PVA-PEG hydrogel or Dextran-PEG hydrogel mix which coats the entire device. In one embodiment, the port size is selected such that the viscosity of the coating polymer is sufficient to prevent it from infiltrating through the ports. The agent 118 is preferably injected through a fine micro-needle which is sufficiently small, such as 3-10 microns, such that the seal coating will re-seal once the needle is inserted and removed from a port. Once the device 110 is completed, it can be packaged into a tube or gun assembly that allows it to be quickly pushed or injected through an appropriately sized conduit disposed near the lesion. Since the device shown in FIG. 2 utilizes a sealing coating, a mechanical seal as discussed for the device 100 in FIG. 1 is not necessary to prevent the sealant from migrating into the agent, or the agent from migrating out of the shell 112. However, a seal may also be incorporated on this embodiment. Though microfabrication and/or 3D printing processes are discussed, traditional methods of manufacture may also be used.

The shell 102 or 112 can have a variety of shapes and sizes that are generally injectable or pushable through a catheter, such as a microcatheter, with an inner diameter from about 0.010-0.027 inches or a guide catheter with an inner diameter from about 0.027-0.130 inches. The approximate diameter (diameter in this context is used broadly since the shell need not be spherical) of the shell is about 20-5000 microns, with the range of 20-1000 microns particularly preferred for delivery through a microcatheter.

The shell 102 or 112 can be made from a variety of materials including glass, polymers such as hydrogels, nylon, PEEK, polyethylene, polyimide, and the like; or metals or their alloys such as platinum, palladium, tantalum, tungsten, steel, and nickel alloys such as nickel-titanium or nickel-cobalt or nickel-chromium. Particularly preferred for some embodiments are palladium or palladium alloys because they combine radiopacity, biocompatibility, corrosion resistance, reasonable cost, and the ability to form radioactive palladium isotopes, such as palladium-103, for certain treatment applications.

The shell 102 or 112 may have a variety of shapes such as spherical, spheroid, pellet-shaped, cylindrical, ellipsoid, cube, and similar shapes. The shell may be formed from a variety of techniques such as blow molding, casting, lost wax casting, sintering powdered metals or plastics, micro machining, 3D printing, 3D photolithography, microfabrication, MEMS technology, etching, plating, multilayer electrochemical fabrication, or a combination of these and similar techniques. Processes described by U.S. Pat. Nos. 7,674,361, 7,368,044, 7,368,044, 7,384,530, 7,271,888, 7,235,166, 7,198,704, 7,527,721, 7,524,427, 8,475,458, 8,613,846, 7,531,077 may also be used and these references and are all hereby incorporated by reference in their entirety.

In some embodiments, the shell 102 or 112 incorporates one or more ports 104 or 114, as previously discussed. The ports are holes, cut-outs, elongated slots, or other features that allow the shell to be at least partially filled with the agent. The size, number, and shape of the port(s) depends on several factors including the fabrication method, the filling apparatus, desired reaction kinetics, and whether or not a seal is incorporated. For example, one or more small (e.g. less than 20% of the shell's surface area) ports may be incorporated when the surface tension of the agent alone is used to hold the agent within the shell or when the desired diffusion of the agent is intended to be relatively slow to allow, for example, prolonged exposure of a tumor to the agent. Conversely, the port(s) may be larger when a seal is incorporated and/or the diffusion of the agent is intended to be faster. The number of ports and/or size of ports can thus be tailored to control the diffusion of the agent. The port(s) may be formed by a variety of techniques such as laser drilling, mechanical drilling, selective etching, or they may be formed at the same time as the shell using, for example, 3D printing or electrochemical fabrication.

The term “agent” should be broadly understood as a term widely encompassing therapeutic and diagnostic materials such as chemotherapy drugs, anti-cancer agents, monoclonal antibodies, proteins, radioactive materials, and the like. The agent (or composition of multiple agents mechanically mixed or chemically bonded to each other) may be a liquid, solid, powder, slurry, oil, or a combination thereof. Non-limiting examples of agents may include chemotherapy drugs such as topoisomerase inhibitors like irinotecan, cytotoxic antibodies such as doxorubicin, platinum-based antineoplastic drugs such as cisplatin, carboplatin, and oxyplatin; anti-microtubule agents such as paclitaxel, or anti-metabolites such as methotrexate. Other non-limiting examples of agents may include monoclonal antibodies such as Campath, Avastin, Erbitux, Zevalin, Arzerra, Vectibix, Rituxam, Bexxar, or Herceptin. Other non-limiting examples of agents include radioactive materials such as palladium-103 chloride, thallium-201 chloride, or iodine-123 useful for therapeutic or diagnostic applications. The agent may also comprise a mixture of drugs, diagnostic materials, and/or radioactive materials. The agent may also comprise an agent mixed with a solvent such as water, DMSO, acetone, or oil such as linseed oil.

In some embodiments, the agent is forced out of the shell by diffusion. Therefore, it may be desirable to dilute or mix the agent with, for example, saline solution or lactated Ringer's solution to bring the agent's salt or pH-level closer to blood in order to slow its diffusion and thus control the agent's release time in the body. Controlling the diffusion rate can also be achieved by adjusting the concentration of the agent to speed or slow its release and uptake.

One embodiment uses carboplatin as the agent because it has been shown to be useful in many types of cancers and currently has no embolic-based delivery system commercially available. Another embodiment uses Avastin because it is a VEGF inhibitor that slows the ability of a tumor to form new blood vessels. This is a highly desirable combination because it is synergistic with the embolic effect of the shell itself mechanically blocking the blood flow to help starve the tumor of blood.

There are a variety of methods for inserting the agent into the shell. For liquid agents, filling can be accomplished with a micro-needle, syringe, micro-pipette, or pump. In some embodiments, standard 30-50 gauge microinjection needles or 5-40 IVF micropipettes may be used. The shells may be arranged on a standardized frame and filled by a computer-controlled filling apparatus. In some cases, it may be desirable to taper the port to match the taper of the filling instrument to ensure proper filling. For solid agents, the shell can be formed around a sintered agent by, for example, 3D printing.

In some embodiments, the surface tension (for liquids) of the agent or other mechanisms are used to hold the agent within the shell. In other embodiments, a seal, plug, or coating (note: the term “seal” should be construed broadly and can cover any of these structures) is used to hold the agent within the shell and/or to protect the agent during manufacturing, packaging, shipping, preparation, and/or delivery. The seal may be made from a variety of materials. Non-limiting examples include biodegradable hydrogels, polylactic acid, polyglycolic acid, sugar, salt, or metals that corrode in the body—such as iron. The speed of the seal's dissolution and, thus, the agent's release, is dependent on the material selection, thickness of the seal, and surface area. The speed of the agent's release can also be controlled by controlling the thickness of the degradable seal.

In one embodiment, the seal selectively disintegrates in proximity to cancer cells or tumors, but remains intact or degrades at a slower rate near other tissues. This may avoid collateral damage to healthy tissue since the seal(s) of device(s) that were not near the tumor would remain substantially intact and thus not release the agent. In this way, the seal can be thought of as a “proximity fuse” which selectively degrades solely in proximity of cancer cells or tumors but not around normal or healthy tissue. Cancer cells have several unique properties that can be used to make this type of “proximity fuse”. For example, many tumors exhibit the Warburg effect in which the cells produce energy by a very high rate of glycolysis and lactic acid fermentation rather than mitochondrial oxidation of pyruvate to ATP as happens in normal cells. As a result, tumors exhibit a high concentration of the dimeric form of the pyruvate kinase enzyme (Tumor M2-PK) which catalyzes energy production by degradation of glutamine (glutaminolysis). Thus, cancer cells have a high affinity for glutamine. In this example, a seal could be a hydrogel made from cross-linked peptides containing glutamine so that the seal would degrade at a higher rate near cancer cells than near normal cells.

Several advantages are offered from the embodiments disclosed herein as compared to traditional methods of delivering anti-cancer drugs. The following is a non-exhaustive, illustrative list. One advantage is that a wide variety of anti-cancer drugs and/or chemotherapy agents can be delivered without having to engineer a polymer structure amenable to bonding each drug/agent. Another advantage is that the drug delivery kinetics can be easily controlled by chanting the size of the port(s) in the structure and the sealing material so that the agent can be delivered over the time scale suited to the patient's disease and the agent being used. Another advantage is that, depending on the material selected for the shell, the device can be radiopaque and thus visible with imaging equipment such as a fluoroscope, CT scanner, MRI scanner, or the like. Another advantage is that, depending on the materials selected, the shell can be radioactive while holding a chemotherapy and/or anti-cancer agent, thus allowing delivery of a combination of therapies in a single device. Another advantage is that the seal can be configured to release a therapeutic or diagnostic agent at a higher rate when in proximity to cancer cells then when near normal tissues, thus avoiding collateral damage to non-target tissues.

Different variations of the drug-delivery devices shown and described herein are contemplated. For instance, several drug delivery devices may be loaded and conveyed to a treatment site in the vasculature. In one embodiment, a series of drug delivery devices may be connected together as part of one drug delivery device for use in the vasculature. The figures and examples offered herein are meant to be illustrative and not limiting.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A drug delivery device, comprising:
a shell sized for passage through a tubular delivery device and into a vessel of a patient; said shell having a cavity and at least one port opening between said cavity and an outside of said shell; and,
a cancer treatment agent disposed within said cavity.

2. The drug delivery device of claim 1, further comprising a seal positioned in said at least one port.

3. The drug delivery device of claim 1, further comprising a sealing coating disposed over said shell and covering said at least one port.

4. The drug delivery device of claim 2, wherein said seal is composed of biodegradable material.

5. The drug delivery device of claim 3, wherein said sealing coating is composed of biodegradable material.

6. The drug delivery device of claim 1, wherein said shell is spherical or ovaloid.

7. The drug delivery device of claim 1, wherein an outer surface of said shell includes a flat portion.

8. The drug delivery device of claim 1, wherein said shell has a diameter between about 20-5000 microns.

9. The drug delivery device of claim 1, wherein said port has a diameter of about 20 microns.

10. The drug delivery device of claim 1, wherein said shell is composed of glass, hydrogel, nylon, PEEK, polyethylene, polyimide, platinum, palladium, tantalum, tungsten, steel, nickel-titanium, nickel-cobalt, or nickel-chromium.

11. The drug delivery device of claim 1, wherein said device is located in a carrier solution and wherein said device and said carrier solution are contained syringe.

12. The drug delivery device of claim 1, wherein said device is located in a microcatheter.

13. The drug delivery device of claim 1, wherein said port further comprises a plurality of ports.

14. The drug delivery device of claim 3, wherein said sealing coating re-seals after a 3-10 micron diameter syringe needle is inserted and removed from said at least one port.

15. The drug delivery device of claim 1, wherein said shell is formed from blow molding, casting, lost wax casting, sintering powdered metals or plastics, micro machining, 3D printing, 3D photolithography, microfabrication, MEMS technology, etching, plating, multilayer electrochemical fabrication, or a combination of these and similar techniques.

16. A method of creating a drug delivery device comprising:
forming a shell having a cavity;
forming a port into a cavity of said shell;
inserting a needle into said port and injecting a cancer treatment agent into said cavity of said shell.

17. The method of claim 16, further comprising plugging said port with a degradable seal.

18. The method of claim 16, further comprising applying a sealing coating to an outer surface of said shell.

19. The method of claim 16, wherein said forming said shell further comprises laser cutting a solid portion of material into said shell shape.

20. The method of claim 16, wherein said forming said shell further comprises casting said shell shape.
